(19) United States
(12) Patent Application Publication
(10) Pub. No.: US 2013/0071904 A1
(43) Pub. Date: Mar. 21, 2013

(54) ACETATE-RESISTANT YEAST STRAIN FOR THE PRODUCTION OF A FERMENTATION PRODUCT

(71) Applicant: Yoram Barak, Menlo Park, CA (US)

(72) Inventor: Yoram Barak, Menlo Park, CA (US)

(21) Appl. No.: 13/578,042

(22) PCT Filed: May 27, 2011
(86) PCT No.: PCT/US11/38311

§ 371 Date: Aug. 9, 2012

Related U.S. Application Data
(60) Provisional application No. 61/349,636, filed on May 28, 2010.
(60) Provisional application No. 61/416,233, filed on Nov. 22, 2010.

(51) Int. Cl. C12P 7/10

(52) U.S. Cl. CPC C12P 7/10
USPC 435/165

(57) ABSTRACT

The present invention provides methods and compositions for fermentations comprising acetate-resistant yeast. The present invention provides methods for use of acetate-resistant yeast for the production of fermentation products.

### FIELD OF THE INVENTION

The present invention provides methods and compositions for fermentations comprising acetate-resistant yeast. The present invention provides methods for use of acetate-resistant yeast for the production of fermentation products.

### BACKGROUND

Ethanol and ethanol fuel blends are widely used in Brazil and in the United States as a transportation fuel. Combustion of these fuels is believed to produce fewer of the harmful exhaust emissions (e.g., hydrocarbons, nitrogen oxide, and volatile organic compounds (VOCs)) that are generated by the combustion of petroleum. Bioethanol is a particularly favored form of ethanol because the plant biomass from which it is produced utilizes sunlight, an energy source that is renewable. In the United States, ethanol is used in gasoline blends that are from 5% to 85% ethanol. Blends of up to 10% ethanol (E10) are approved for use in all gasoline vehicles in the U.S. and blends of up to 85% ethanol (E85) can be utilized in specially engineered flexible-fuel vehicles (FFV). The Brazilian government has mandated the use of ethanol-gasoline blends as a vehicle fuel, and the mandatory blend has been 25% ethanol (E25) since 2007.

Bioethanol is currently produced by the fermentation of hexose sugars that are obtained from carbon feedstocks. Currently, only the sugar from sugar cane and starch from feedstock such as corn can be economically converted. There is, however, much interest in using lignocellulosic feedstocks where the cellulose part of a plant is broken down to sugars and subsequently converted to ethanol. Lignocellulosic biomass is made up of cellulose, hemicelluloses, and lignin. Cellulose and hemicellulose can be hydrolyzed in a saccharification process to sugars that can be subsequently converted to ethanol via fermentation. The major fermentable sugars from lignocelluloses are glucose and xylose.

### SUMMARY OF THE INVENTION

The present invention provides methods and compositions for fermentations comprising acetate-resistant yeast. The present invention provides methods for use of acetate-resistant yeast for the production of fermentation products.

The present invention provides methods for fermentation comprising: providing at least one acetate-resistant Saccharomyces cerevisiae NRRL YB-1952 cell or a genetically modified derivative of said Saccharomyces cerevisiae NRRL YB-1952 cell and a fermentation medium comprising acetate; culturing the at least one acetate-resistant Saccharomyces cerevisiae NRRL YB-1952 cell or a genetically modified derivative of said Saccharomyces cerevisiae NRRL YB-1952 cell in the fermentation medium under conditions such that the at least one acetate-resistant Saccharomyces cerevisiae NRRL YB-1952 cell or a genetically modified derivative of said Saccharomyces cerevisiae NRRL YB-1952 cell produces at least one fermentation product. In some embodiments, the fermentation medium comprises acetate at a concentration of at least 6 g/L. In some further embodiments, the methods further comprise collecting the fermentation product. In some additional embodiments, the methods further comprise distilling the fermentation product from the culture medium. In yet some further embodiments, the fermentation medium comprises saccharified lignocellulose. In some embodiments, the fermentation medium comprises lignocellulose feedstock that has been pre-treated. In some additional embodiments, the saccharified lignocellulose is produced by enzymatic and/or acidic treatment of a lignocellulose feedstock. In some embodiments, the fermentation product is an alcohol. In some additional embodiments, the alcohol is ethanol. In still some additional embodiments, the fermentation medium further comprises at least one organic acid at a concentration of at least 6 g/L. In some further embodiments, the fermentation medium has a pH of less than pH 6.0. In some additional embodiments, the methods are conducted under anaerobic conditions. In some further embodiments, the method is a batch-fed fermentation method or a continuous fed fermentation method.

### BRIEF DESCRIPTION OF THE FIGURE

FIG. 1 provides graphs showing glucose consumption and ethanol production in AFM fermentations using concentrated process sugars for strains NRRL YB-1952, NRRL Y7567, SUPERSTART™ dry yeast (Lallemand), and THERMOSACC® fresh yeast (Lallemand) Cell loads at fermentation initiation were normalized at 7 g/L dry cell weight (DCW).

### DESCRIPTION OF THE INVENTION

The present invention provides methods and compositions for fermentations comprising acetate-resistant yeast. The present invention provides methods for use of acetate-resistant yeast for the production of fermentation products.

All patents and publications, including all sequences disclosed within such patents and publications, referred to herein are expressly incorporated by reference. Unless otherwise indicated, the practice of the present invention involves conventional techniques commonly used in molecular biology, fermentation, microbiology, and related fields, which are known to those of skill in the art. Unless defined otherwise herein, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described. Indeed, it is intended that the present invention not be limited to the particular methodology, protocols, and reagents described herein, as these may vary, depending upon the context in which they are used. The headings provided herein are not limitations of the various aspects or embodiments of the present invention.

Nonetheless, in order to facilitate understanding of the present invention, a number of terms are defined below. Numeric ranges are inclusive of the numbers defining the range. Thus, every numerical range disclosed herein is intended to encompass every narrower numerical range that falls within such broader numerical range, as if such narrower numerical ranges were all expressly written herein. It is also intended that every maximum (or minimum) numerical limitation disclosed herein includes every lower (or higher) numerical limitation, as if such lower (or higher) numerical limitations were expressly written herein.

As used herein, the term “comprising” and its cognates are used in their inclusive sense (i.e., equivalent to the term “including” and its corresponding cognates).

As used herein and in the appended claims, the singular “a”, “an” and “the” includes the plural reference unless the context clearly dictates otherwise. Thus, for example, reference to a “host cell” includes a plurality of such host cells.

Unless otherwise indicated, nucleic acids are written left to right in 5′ to 3′ orientation; amino acid sequences are written left to right in amino to carboxy orientation, respectively. The headings provided herein are not limitations of the various aspects or embodiments of the invention that can be had by reference to the specification as a whole. Accordingly, the terms defined below are more fully defined by reference to the specification as a whole.

As used herein, the terms “isolated” and “purified” are used to refer to a molecule (e.g., an isolated nucleic acid, polypeptide, etc.) or other component that is removed from at least one other component with which it is naturally associated.

As used herein, the term “recombinant” refers to a polynucleotide or polypeptide that does not naturally occur in a host cell. A recombinant molecule may contain two or more naturally-occurring sequences that are linked together in a way that does not occur naturally. A recombinant cell contains a recombinant polynucleotide or polypeptide.

As used herein, the term “overexpress” is intended to encompass increasing the expression of a protein to a level greater than the cell normally produces. It is intended that the term encompass overexpression of endogenous, as well as heterologous proteins.

For clarity, reference to a cell of a particular strain refers to a parental cell of the strain as well as progeny and genetically modified derivatives of the same. Genetically modified derivatives of a parental cell include progeny cells that contain a modified genome or episomal plasmids that confer for example, antibiotic resistance, improved fermentation, the ability to utilize xylose as a carbon source, etc.

As used herein, the term “acetate-resistant” refers to fungal strains that are capable of surviving and fermenting sugars in the presence of relatively high acetate concentrations. In some embodiments, fermentation medium used to grow acetate-resistant fungal strains comprises an acetate concentration of about 5 to about 50 g/L acetate. In some embodiments, the acetate concentration is about 10 g/L (1% w/v). In some embodiments, the pH of the medium is about 5.5 or less.

As used herein, the term “lignocellulose” refers to a structural component of plant material that is composed of cellulose, hemicellulose and lignin. Cellulose is a polymer of glucose with beta-1,4 linkages. Hemicellulose has a more complex structure that varies among the different plants. Unlike cellulose, hemicellulose contains pentose sugar (e.g., xylose and/or arabinose). For many plants, hemicellulose is composed of a backbone polymer of xylose residues that are linked together by beta-1,4 linkages and side chains of 1 to 5 arabinose units that are linked together by alpha-1,3 linkages. The side chains may contain acetyl moieties or other organic acid moieties such as glucuronyl groups. Lignin is an insoluble high molecular weight material of aromatic alcohols that provides strength. Lignin generally contains three aromatic alcohols (coniferyl alcohol, sinapyl and p-coumaryl). In addition, grass and dicot lignin also contain large amounts of phenolic acids such as p-coumaric and ferulic acid, which are esterified to alcohol groups of each other and to other alcohols such as sinapyl and p-coumaryl alcohols. Lignin may be linked to both hemicelluloses and cellulose forming a physical seal around the latter two components that is an impenetrable barrier preventing penetration of solutions and enzymes.

As used herein, the term “lignocellulosic feedstock” refers to any type of plant-derived biomass that contains lignocellulose. In certain embodiments a lignocellulosic feedstock may contain at least about 50%, at least about 70% or at least about 90% (by dry weight) lignocellulose. It is understood that lignocellulosic feedstock may also contain other constituents in addition to lignocellulose, such as fermentable sugars, un-fermentable sugars, proteins, oil, carbohydrates, etc. Lignocellulosic feedstock may contain stems, leaves, hulls, husks, and cobs of plants or leaves, branches, and wood of trees, for example. Lignocellulosic feedstock includes herbaceous material, agricultural residue, forestry residue, waste paper, and pulp and paper mill residues. Certain lignocellulosic feedstocks contain about 30% to about 50% cellulose, about 15% to about 35% hemicelluloses, and about 15% to about 30% lignin.

Examples of lignocellulosic feedstock include material from woody plants (which may be softwood or hardwood, for example, poplar or birch) and non-woody plants such as C3 grasses and C4 grasses, including switchgrass, cord grass, rye grass, miscanthus, reed canary grass etc, as well as processed products thereof. The term “lignocellulosic feedstock” also refers to agricultural residues (e.g., soybean stover, corn stover, rice straw, rice hulls, barley straw, corn cobs, wheat straw, canola straw, rice straw, oat straw, oat hulls, corn fiber, as well as woody plant products such as recycled wood pulp fiber, sawdust, newsprint, cardboard, sawdust, etc.).

As used herein, the term “saccharified lignocellulose” refers to lignocellulosic feedstock that has been processed to release sugars that can be fermented to ethanol. The hydrolytic process used to produce saccharified lignocellulose typically includes acid or enzymatically treating a lignocellulosic feedstock to hydrolyze the cellulose and hemicellulose components of the lignocellulose, thereby releasing monomeric sugars. Saccharified lignocellulose contains glucose and at least one pentose sugar (e.g., xylose or arabinose).

As used herein, the terms “ferment”, “fermenting” and “fermentation” refer to a biochemical process in which a carbon source (e.g., a sugar) is broken down to produce at least one fermentation product, including but not limited to such products as alcohols (e.g., ethanol, butanol, etc.), fatty alcohols (e.g., C8-C20 fatty alcohols), acids (e.g., lactic acid, 3-hydroxypropionic acid, acrylic acid, acetic acid, succinic acid, citric acid, malic acid, fumaric acid, amino acids, etc.), 1,3-propane diol, ethylene glycol, glycerol, terpenes, and antimicrobials (e.g., β-lactams such as cephalosporin), etc. In some embodiments in which ethanol is produced by fermentation, other products, including but not limited to lactate, acetic acid, hydrogen and carbon dioxide are also produced.

### Culture Conditions

It is intended that the present invention will find use at any volume desired. Thus, it is not intended that the present invention be limited to culture media of any particular volume.

In some embodiments, the culture medium in which the cells are present comprises saccharified lignocellulose which, as noted above, contains glucose and one or more pentose sugars (e.g., one or more pentose sugars selected from xylose and arabinose). Depending on the feedstock used, the culture medium may also contain mannose, galactose, and/or rhamnose. In some embodiments, the concentration of glucose and pentose sugars in the composition varies, depending on the feedstock used and the growth phase of the culture. In some embodiments, the culture medium comprises glucose at a concentration in the range of about 0.001% to about 50% (w/v) (e.g., about 1% to about 30% (w/v)), although in some embodiments, other glucose concentrations find use. In some embodiments, the culture medium comprises at least one pentose sugar (e.g., xylose and/or arabinose) at a concentration in the range of about 0.001% to about 50% (w/v) (e.g., about 1% to about 30% (w/v)) or more. In some other embodiments, other pentose concentrations find use. In some embodiments, depending on how the feedstock is made, the culture medium comprises lignin and/or lignin-derived products (e.g., phenolics or aromatic alcohols). In some alternative embodiments, the lignin is removed from the culture medium prior to use. Methods for treating lignocellulosic feedstock to provide saccharified lignocellulose are known in the art and described below.

The pH of the culture medium is in the range of about pH 3.0 to about pH 7.0 (e.g., about 3.0, about 3.5, about 4.0, about 4.5, about 5.0, about 5.5, about 6.0, about 6.5, or about 7.0). However, pH ranges below about 3.0 or above about 7.0 find use in some embodiments.

In some embodiments, the fermentation is carried out at a temperature between about 20° C. to about 40° C. (e.g., about 20° C., about 25° C., about 30° C., about 35° C., or about 40° C.).

In some embodiments, the culture is maintained for about 20 to about 96 hours (e.g., about 20 hours, about 21 hours, about 22 hours, about 23 hours, about 24 hours, about 25 hours, about 27 hours, about 30 hours, about 35 hours, about 40 hours, about 45 hours, about 50 hours, about 55 hours, about 60 hours, about 65 hours, about 70 hours, about 75 hours, about 80 hours, about 85 hours, about 90 hours, about hours, or about 96 hours).

In addition, in some embodiments, the culture medium comprises at least one organic acid (e.g., acetate) at a concentration of at least about 3 g/L, at least about 4 g/L, at least about 5 g/L, at least about 6 g/L, at least about 7 g/L, at least about 8 g/L, at least about 9 g/L, at least about 10 g/L, at least about 12 g/L, at least about 15 g/L, at least about 20 g/L, at least about 25 g/L, at least about 30 g/L, at least about 35 g/L, at least about 40 g/L, at least about 45 g/L, at least about 50 g/L, at least about 55 g/L, at least about 60 g/L.

In some embodiments, the cells in the culture are resistant to low pH and high acetic acid conditions relative to other cells (e.g., NRRL Y-7567 or other industrial strains of S. cerevisiae known as SUPERSTART™ and THERMOSACC® (available from Lallemand) In some embodiments, the cells are grown in an aqueous environment under anaerobic conditions (i.e., in the absence of added oxygen) to facilitate fermentation of the sugar in the culture medium by the cells to produce the desired fermentation product (e.g., ethanol). In some embodiments, the culture medium also comprises a fermentation product (e.g., ethanol). In some embodiments, the fermentation product is present at a concentration in the range of about 0.1% to about 50% (w/v), including 1%, about 5%, about 6%, about 7%, about 8%, about 9%, about 10%, about 11%, about 12%, about 13%, about 14%, about 15%, about 20%, about 22%, about 25%, about 27%, about 30%, about 32%, about 35%, about 37%, about 40%, about 42%, about 45%, about 47%, or about 50%. (However, in some embodiments, the fermentation product is present at a concentration of less than about 0.1% or more than about 50%. In some embodiments, the culture medium comprises acetate at a concentration in the range of about 6 g/L to about 12 g/L and have a pH in the range of about pH 4.0 to about pH 6.0.

In some embodiments, the S. cerevisiae cells are capable of faster growth, produce more of a fermentation product (e.g., ethanol), and utilize more glucose than NRRL Y-7567, SUPERSTART™ dry yeast or THERMOSACC® fresh yeast cells grown in a culture that contains acetate at a concentration in the range of about 6 g/L to about 12 g/L having a pH in the range of about pH 4.0 to about pH 6.0. Growth rate can be determined by any suitable method known in the art (e.g., optical density, cell counting methods, and other well known methods). In some embodiments, the S. cerevisiae cells produce ethanol at a rate of at least about 0.5 g/L/h, (e.g., at least about 1.0 g/L/h, at least about 2.0 g/L/h, at least about 3.0 g/L/h, at least about 5.0 g/L/h, at least about 10 g/L/h, at least about 15 g/L/h, at least about 20 g/L/h, at least about 25 g/L/h , at least about 30 g/L/h, at least about 35 g/L/h, at least about 40 g/L/h, at least about 45 g/L/h, at least about 50 g/L/h, at least about 60 g/L/h, at least about 70 g/L/h, at least about 80 g/L/h, or at least about 90 g/L/h, up to at least about 100 g/L/h) in culture medium containing saccharified lignocellulose. In some embodiments, the S. cerevisiae cells utilize glucose a rate of at least about 10 g/L/h (e.g., at least about 20 g/L/h, at least about 30 g/L/h, at least about 40 g/L/h, at least about 50 g/L/h, at least about 100 g/L/h, at least about 200 g/L/h, at least about 300 g/L/h, at least about 400 g/L/h, up to at least about 500 g/L/h) in culture medium containing saccharified lignocellulose. In some embodiments, the S. cerevisiae cells utilize glucose at a rate that is about twice the product (e.g., ethanol) production rate. In some embodiments, the S. cerevisiae cells are genetically modified to ferment at least one pentose sugar. In some embodiments, the cell comprises a recombinant nucleic acid for the expression of one or more enzymes selected from xylose isomerase, xylose reductase, xylitol dehydrogenase and/or xylitol isomerase. Cells that are genetically modified to ferment pentose sugar are discussed in greater detail below.

In embodiments, fermentation of glucose and other carbon sources by S. cerevisiae cells produces acetate as a by-product. Likewise, the processing of lignocellulose feedstock into saccharified lignocellulose can employ or produce acetate. Acetate, however, inhibits the growth of Saccharomyces cells (See e.g., Pons et al., Appl. Microbiol. Biotech., 3: 193-198 [1984]). Thus, the fermentation of saccharified lignocellulose by S. cerevisiaeis is often inefficient. The S. cerevisiae provided herein are more resistant to acetate than other S. cerevisiae strains. Thus, this strain finds use in fermentation methods that are conducted in the presence of acetate, including fairly high concentrations of acetate.

### Engineered Cells

The present invention also provides a recombinant S. cerevisiae cells comprising a recombinant polynucleotide. In some embodiments, this polynucleotide is operatively linked to its native promoter, or to a heterologous promoter (i.e., one not associated with the polynucleotide in the corresponding native gene) to, for example, overexpress the polynucleotide. In some embodiments, the recombinant S. cerevisiae cells optionally comprise multiple copies of the polynucleotide. Suitable polynucleotides include those which facilitate overexpression of proteins known to have an impact on the desired phenotype. Therefore, in some embodiments, the Saccharomyces cerevisiae cells are altered or engineered to overexpress one or more poylnucleotides.

In some embodiments, the recombinant S. cerevisiae cells comprise a recombinant polynucleotide that confers the ability to ferment a pentose sugar (e.g., to provide for conversion of xylose into ethanol) is also provided. As noted above, in some embodiments, the cells comprise a recombinant polynucleotide that encodes an enzyme selected from a xylose isomerase, a xylose reductase, a xylitol dehydrogenase, a xylulokinase, a xylitol isomerase and/or a xylose transporter. Strategies for genetically modifying S. cerevisiae cells to ferment pentose sugars (particularly xylose) are known by those of skill in the art (See e.g., Matsushika, Appl. Microbiol. Biotechnol., 84:37-53 [2009]; van Maris, Adv. Biochem. Eng., Biotechnol. 108:179-204 [2007]; Hahn-Hägerdal, Adv. Biochem. Eng. Biotechnol., 2007 108: 147-177 [2007]; and Jeffries, Curr. Opin. Biotechnol., 17:320-3266 [2006]).

Suitable methods involve heterologous expression of xylose isomerase, optionally in combination with xylulokinase, in S. cerevisiae cells (See e.g., Brat, Appl. Environ. Microbiol., 75:2304-11 [2009]); Madhavan, Appl. Microbiol. Biotechnol., 82: 1067-78 [2009]; and Kuyper, FEMS Yeast Res., 4:69-78 [2003]) and heterologous expression of xylitol dehydrogenase and xylose reductase in S. cerevisiae cells (See e.g., Krahulec, Biotechnol. J., 4: 684-694 [2009]; Bettiga, Biotechnol. Biofuels 1:16 [2008]; and Matsushika, J. Biosci. Bioeng., 105:296-299 [2008]), alone or in combination with other components of the pentose catabolism or sugar uptake pathways, and/or other ethanologenic enzymes (e.g., pyruvate decarboxylase, aldehyde dehydrogenase, and/or an alcohol dehydrogease) and/or various other genetic modifications.

Recombinant polynucleotides that encode xylose isomerases which are suitable for use in the present invention include, but are not limited to the xylose isomerase genes from Clostridium phytofermentans (Genbank Accession No. ABX41597.1), Piromyces sp. E2 (Genbank Accession No. CAB76571.1), Clostridium, Fusobacter, Saccharomyces, Kluyveromyces, Candida, Picha, Schizosaccharomyces, Ruminococcus flavefaciens, Hansenula, Kloeckera, Schwanniomyces, Yarrowia Aspergillus, Trichoderma, Humicola, Acremonium, Penicillium, etc. (See e.g., WO 2010/074577, which is incorporated herein by reference), Pseudomonas syringae (See e.g., WO 2010/070549, which is incorporated herein by reference), Thermoanaerobacter thermohydrosulfuricus (See e.g., WO 2010/070549, which is incorporated herein by reference), Thermoanaerobacter thermohydrosulfurigenes (See e.g., WO 2010/070549, which is incorporated herein by reference), and Lactococcus lactis susp. lactis (Lactobacillus xylosus) (See e.g., WO 2010/070549, which is incorporated herein by reference).

Recombinant polynucleotides that encode transporters which are suitable for use herein include those that are well known in the art, such as, for example, GXF1, SUT1 and At6g59250 from Candida intermedia, Pichia stipitis, and Arabidopsis thaliana, respectively (See e.g., Runquist et al., Biotechnol Biofuels 3:5 [2010], which is incorporated herein by reference). Also suitable are transporters, including, but not limited to HXT4, HXT5, HXT7, GAL2, AGT1, and GXF2 (See e.g., Matsushika et al., Appl. Microbiol. Biotechnol., 84:37-53 [2009], which is incorporated herein by reference). In some embodiments, overexpression of the native S. cerevisiae transporters is desirable, particularly HXT5 and HXT7. Therefore, in some embodiments, the recombinant S. cerevisiae host cells comprise at least one heterologous promoter operably linked to a polynucleotide encoding HXT5 and/or HXT7.

Other suitable recombinant polynucleotides that find use in the present invention include, but are not limited to those that encode: at least one xylulose kinase (XK); at least one enzyme from the pentose phosphate pathway (e.g., a ribulose-5-phosphate 3-epimerase (RPE1), a ribose-5-phosphate keto-isomerase (RKI1), a transketolase (TKL1), a transaldolase (TAL1), and the like); at least one enzyme from the glycolysis metabolic pathway (e.g., a hexokinase (HXK1/HXK2), a glyceraldehyde-3-phosphate dehydrogenase (GAPDH), a pyruvate kinase (PVK2), and the like); and/or at least one ethanologenic enzyme (e.g., pyruvate decarboxylase and/or an alcohol dehydrogenase).

Recombinant polynucleotides that are suitable for use herein also include regulatory polynucleotides (e.g., heterologous regulatory polynucleotides), including, but not limited to promoters, enhancers, and/or terminators, as well as other regulatory elements that function to improve the expression of polynucleotides in S. cerevisiae cells.

The present invention also provides engineered S. cerevisiae cells in which one or more of the native genes have been deleted from its genome and/or one or more native genes have been inactivated. In some embodiments, the deletion(s) cause removal or diminishment of a biological activity that is otherwise exhibited by the cells. In some embodiments, the cumulative effect of the deletion(s) results in an improvement in a phenotype of the S. cerevisiae cells. Any suitable method for deleting or inactivating genes in S. cerevisiae finds use in the present invention.

For example, in some embodiments, engineered Saccharomyces cerevisiae cells have at least one of their native genes deleted from the host genome in order to improve the utilization of pentose sugars (e.g., xylose, arabinose, etc.), increase transport of xylose into the cell, increase xylulose kinase activity, increase flux through the pentose phosphate pathway, decrease sensitivity to catabolite repression, increase tolerance to ethanol, increase tolerant to acetate, increase tolerance to increased osmolarity, increase tolerance to organic acids (low pH), reduce production of by products, and other like properties related to increasing flux through the relevant pathways to produce ethanol and other desired metabolic products at higher levels, where comparison is made with respect to the corresponding cell without the deletion(s). Genes targeted for deletion include, for example, genes encoding the enzymes in the pentose phophate pathway, glycolysis, and/or pathways involved in the production of ethanol.

In some additional embodiments, other genes that are targeted for deletion include those encoding, for example, aldose reductase (GRE3) (See, Matsushika et al., Appl. Microbiol. Biotechnol., 84:37-53 [2009]), sorbitol dehydrogenase (SOR1/SOR2), glutamate dehydrogenase (GDH1), 6-phosphogluconate dehydrogenase (GND), glucose-5-phosphate dehydrogenase (ZWF1), and any enzyme in which its deletion is known in the art to improve the utilization of a pentose sugar, decrease by product formation, and/or increase the ethanol yield of the engineered Saccharaomyces cerevisiae. Those of ordinary skill in the art appreciate that additional genes encoding these enzymes can be readily identified by microarray analysis (See e.g., Sedlak et al., Yeast 21:671-684 [2004]), metabolic flux analysis (See e.g., Sonderegger et al., Appl. Environ. Microbiol., 70:2307-2317 [2004]), in silico modeling (See e.g., Hjersted et al., Biotechnol. Bioengineer., 97:1190-1204 [2007]), chemogenomics (Teixeira et al., Appl. Environ. Microbiol. 75:5761-5772 [2009]) and other well known methods.

Methods for recombinant expression of proteins in yeast are well known in the art, and a number of vectors are available or can be constructed using routine methods (See e.g., Tkacz and Lange, Advances in Fungal Biotechnology for Industry, Agriculture, and Medicine, Kluwer Academic/Plenum Publishers, New York [2004]; Zhu et al., Plasmid 6:128-33 [2009]; and Kavanagh, Fungi: Biology and Applications, John Wiley & Sons, Malden, M A [2005]; all of which are incorporated herein by reference).

In some embodiments, recombinant nucleic acid constructs for use in yeast further contain a transcriptional regulatory element that is functional in a yeast cell. In some embodiments, the nucleic acid construct comprises polynucleotide operatively linked to a transcriptional regulatory sequence (e.g., a promoter, a transcription termination sequence, etc.), that is functional in a yeast cell. Promoters that are suitable for use include endogenous or heterologous promoters and include both constitutive and inducible promoters that are natural or modified. Particularly useful promoters are those that are insensitive to catabolite (glucose) repression and/or do not require xylose or glucose for induction. Such promoters are well known in the art.

Promoters that are suitable for use herein include, but are not limited to yeast promoters from glycolytic genes, (e.g., yeast phosphofructokinase (PFK), triose phosphate isomerase (TPI), glyceraldehyde-3-phosphate dehydrogenase (GPD, TDH3 or GAPDH), pyruvate kinase (PYK), glucose transporters; ribosomal protein encoding gene promoters; alcohol dehydrogenase promoters (ADH1, ADH4, etc.), enolase promoter (ENO), phosphoglycerate kinase (PGK), etc.; See e.g., WO 93/03159, which is incorporated herein by reference);

Exemplary promoters that are useful for directing the transcription of the nucleic acid constructs in yeast host cells include those from the genes for S. cerevisiae enolase (eno-1), S. cervisiae galactokinase (gall), S. cerevisiae alcohol dehydrogenase/glyceraldehyde-3-phosphate dehydrogenase (ADH1/ADH2/GAP), S. cerevisiae 3-phosphoglycerate kinase, S. cerevisiae, transcription elongation factor (TEF), S. cerevisiae fructose 1,6-bisphosphate aldolase (FBA1), and S. cerevisiae 3-phosphate glycerate kinase (PGK1). Other useful promoters for yeast host cells are well known in the art (See e.g., Romanos et al., Yeast 8:423-488 [1992], incorporated herein by reference).

Exemplary transcription termination sequences (terminators) that are functional in a yeast host cell include transcription termination sequences from yeast cells. Exemplary yeast transcription termination sequences include those of the CYC1, ADH1t and ADH2t genes, etc. In some embodiments, the nucleic acid constructs optionally contain a ribosome binding site for translation initiation. The constructs also optionally include appropriate sequences for amplifying expression (e.g., an enhancer). Such elements are well known in the art.

In addition, in some embodiments, the nucleic acid constructs contain one or more selectable marker genes to provide a phenotypic trait for selection of transformed cells. Suitable marker genes include, but are not limited to those coding for resistance to antibiotics or antimicrobials (e.g., ampicillin, kanamycin, chloramphenicol, tetracycline, streptomycin, spectinomycin, neomycin, geneticin, nourseothricin, hygromycin, and/or phleomycin), as other marker genes that are well known in the art. In some embodiments, the nucleic acid constructs contain a yeast origin of replication. Examples include constructs containing autonomous replicating sequences, constructs containing 2 micron DNA including the autonomous replicating sequence and rep genes, constructs containing centromeres like the CEN6, CEN4, CEN11, CDN3 and autonomous replicating sequences, and other like sequences that are well known in the art. Exemplary nucleic acid constructs include constructs suitable for transforming yeast. These include episomal constructs based on the yeast 2μ or CEN origin based plasmids like pYES2/CT, pYES3/CT, pESC/His, pESC/Ura, pESC/Trp, pESC/Leu, p427TEF, pRS405, pRS406, pRS413, and other yeast-based constructs known in the art.

In some embodiments, the nucleic acid constructs comprise elements to facilitate integration of a heterologous polynucleotide into a fungal genome by site-directed or random homologous or non-homologous recombination. In some embodiments, the nucleic acid constructs comprise elements which facilitate homologous integration. In some embodiments, the polynucleotide is integrated at one or more sites, to provide one or more copies of the sequence in the genome. In some embodiments, the nucleic acid constructs comprise a protein-coding polynucleotide and a promoter that is operatively linked to the polynucleotide. This type of construct typically comprises genetic elements to facilitate integration into the fungal chromosome at a location that is downstream of a native promoter (i.e., in the host chromosome). Alternatively, in some embodiments, a second nucleic acid construct employed which comprises a promoter and genetic elements to facilitate integration into the fungal genome in a location upstream of the targeted integration site of the polynucleotide.

Genetic elements that facilitate integration by homologous recombination include those having sequence homology to targeted integration sites in the fungal genome. Suitable sites that find use as targets for integration include, for example, the TY1 locus, the RDN locus, the ura3 locus, the GPD locus, aldose reductase (GRE3) locus, etc. Those of skill in the art appreciate that additional sites for integration can be readily identified by microarray analysis, metabolic flux analysis, comparative genome hybridization analysis, and other such methods that are well known in the art.

Genetic elements or techniques which facilitate integration by non-homologous recombination include restriction enzyme-mediated integration (REMI) (See e.g., Manivasakam et al., Mol. Cell Biol., 18:1736-1745 [1998], incorporated herein by reference), transposon-mediated integration, as well as additional elements and methods well known in the art.

Cultivation, transformation and selection of a transformed yeast cell and also expression of a protein in a yeast cell are among the methods commonly used by those of skill in the art and are described in many texts and other references. In addition, in some embodiments, cells are optionally mutagenized and/or evolved to exhibit further desired phenotypes (e.g., for further improvement in the utilization of glucose and/or pentose sugars, increased transport of sugar into the host cell, increased flux through the pentose phosphate pathway, decreased sensitivity to catabolite repression, increased tolerance to ethanol, increased tolerance to acetate, increased tolerance to increased osmolarity, increased tolerance to organic acids (low pH), reduced production of byproducts, etc.).

### Method for Conferring Acetate Resistance to Cells

The present invention also provides methods for conferring resistance to acetate in cells. In some embodiments, the method comprises: a) identifying an acetate-resistance locus in an acetate-resistant microbial strain; and b) transferring the locus to a non-acetate-resistant strain, such that acetate-resistance is imparted to the previously non-acetate-resistant strain. This new acetate-resistant strain finds use in production of at least one fermentation product under conditions in which the strain is exposed to high acetate concentrations. In some embodiments, the new strain produces ethanol (or desirable other end-product(s)) using a culture medium comprising saccharified lignocellulose, as described above. In some embodiments, the acetate-resistant strain is NRRL YB-1952. In some additional embodiments, the acetate-resistant strain produces high ethanol concentrations compared with strains that are not acetate-resistant.

In some embodiments, the genome NRRL YB-1952 is fragmented, the fragments cloned into a vector, and the vector transferred into a non-acetate-resistant S. cerevisiae strain (e.g., NRRL Y-7567 or other industrial strains of S. cerevisiae such as SUPERSTART™ dry yeast or THERMOSACC® fresh yeast ([commercially available from Lallemand Ethanol Technology, Milwaukee, Wis.]). The resultant library of cells are plated and tested for acetate-resistance, in order to identify cells that contain a vector that confers acetate resistance. Methods for isolating dominant and recessive loci from S. cerevisiae have been successfully performed for decades, and such methods may be readily adapted for use in the present invention (See e.g., van den Berg, Yeast 13: 551-559 [1997]).

### Method for Making a Fermentation Product

The present invention also provides methods for making a fermentation product (e.g., ethanol). In general terms, the methods comprise maintaining the above-described cell culture under conditions suitable for the production of the fermentation product. In these methods, the sugar present in the cell culture is fermented by the cells to produce at least one fermentation product. In some embodiments, the fermentation product(s) is collected from the culture. In some additional embodiments, the methods comprise distilling the fermentation product from the culture using methods known in the art.

In some embodiments, the methods comprises producing saccharified lignocellulose by acid or enzymatic treatment of a lignocellulose feedstock, thereby producing a product comprising glucose and at least one pentose sugar selected from xylose and arabinose; and contacting a S. cerevisiae cell with the saccharified lignocellulose to produce the fermentation product.

In some embodiments, prior to contacting the saccharified lignocellulose with a S. cerevisiae cell, the lignocellulose feedstock is treated to release monomeric sugars. In some embodiments, the lignocellulose feedstock is hydrolyzed (e.g., by acid treatment or enzymatically), before and/or during fermentation to saccharify the cellulose and hemicellulose.

In some embodiments, the first process step for converting lignocellulosic feedstock to a fermentation product involves breaking down (i.e., depolymerizing) the fibrous material. The two primary processes are acid hydrolysis, which involves the hydrolysis of the feedstock using a single step of acid treatment, and enzymatic hydrolysis, which involves an acid pretreatment followed by hydrolysis with cellulase enzymes.

In some embodiments, the feedstock is treated with an acid. In such embodiments, the feedstock is subjected to steam and an acid (e.g., a mineral acid such as sulfuric acid, sulfurous acid, hydrochloric acid, or phosphoric acid). The temperature, acid concentration and duration of the acid hydrolysis are sufficient to hydrolyze the cellulose and hemicellulose to their monomeric constituents (i.e., glucose from cellulose and xylose and one or more of galactose, mannose, arabinose, acetic acid, galacturonic acid, and glucuronic acid from hemicelluloses). In some embodiments in which sulfuric acid is utilized, it can be utilized in concentrated (about 25-about 80% w/w) or dilute (about 3 to about 8% w/w) form. The resulting aqueous slurry contains unhydrolyzed fiber that is primarily lignin, and an aqueous solution of glucose, xylose, organic acids, including primarily acetic acid, as well as glucuronic acid, formic acid, lactic acid and galacturonic acid, and the mineral acid.

In some other embodiments, the feedstock is treated with steam, mild acid and an enzyme. In these embodiments, the steam temperature, acid (e.g., a mineral acid such as sulfuric acid) concentration and treatment time of the acid pretreatment step are chosen to be milder than that in the acid hydrolysis process. Similar to the acid hydrolysis process, the hemicellulose is hydrolyzed to one or more of xylose, galactose, mannose, arabinose, acetic acid, glucuronic acid, formic acid, and/or galacturonic acid. However, the milder pretreatment does not hydrolyze a large portion of the cellulose, but rather increases the cellulose surface area as the fibrous feedstock is converted to a muddy texture. The pretreated cellulose is then hydrolyzed to glucose in a subsequent step that uses cellulase enzymes.

In some embodiments, prior to the addition of enzyme, the pH of the acidic feedstock is adjusted to a value that is suitable for the enzymatic hydrolysis reaction. In some embodiments, this involves the addition of alkali to a pH of between about 4 and about 6, which is the optimal pH range for cellulases, although the pH can be higher if alkalophilic cellulases are used and lower if acidic cellulases are used. Solutions that are most commonly used to adjust the pH of the acidified pretreated feedstock prior to hydrolysis by cellulase enzymes include ammonia, ammonium hydroxide and sodium hydroxide, although the use of carbonate salts such as potassium carbonate, potassium bicarbonate, sodium carbonate and sodium bicarbonate can also be used.

In some embodiments, at least three categories of enzymes are used to convert cellulose into glucose: endoglucanases (EC 3.2.1.4) that cleave the cellulose chains at random positions; cellobiohydrolases (EC 3.2.1.91) which cleave cellobiosyl units from the cellulose chain ends; and beta-glucosidases (EC 3.2.1.21) that convert cellobiose and soluble cellodextrins into glucose.

In some embodiments, after contacting the saccharified lignocellulose with the subject cell, the culture is maintained under suitable conditions (i.e., time, temperature and pH, etc.) for the production of ethanol by the cell. Fermentation conditions suitable for generating ethanol are well known in the art. In some embodiments, the fermentation process is carried out under aerobic conditions, while in other embodiments microaerobic (i.e., where the concentration of oxygen is less than that in air) or anaerobic conditions are used. Typical anaerobic conditions are the absence of oxygen (i.e., no detectable oxygen), or less than about 5, about 2.5, or about 1 mmol/L/h oxygen. In the absence of oxygen, the NADH produced by glycolysis cannot be oxidized by oxidative phosphorylation. Under anaerobic conditions, pyruvate or a derivative thereof may be utilized by the host cell as an electron and hydrogen acceptor in order to generated NAD+. In some embodiments, when the fermentation process is carried out under anaerobic conditions, pyruvate is reduced to at least one fermentation product, including but not limited to ethanol, butanol, fatty alcohol (e.g., C8-C20 fatty alcohols), lactic acid, 3-hydroxypropionic acid, acrylic acid, acetic acid, succinic acid, citric acid, malic acid, fumaric acid, an amino acid, 1,3-propanediol, ethylene, glycerol, terpenes, and/or antimicrobials (e.g., β-lactams, such as cephalosporin).

In some embodiments, the fermentation involves batch processes, while in other embodiments, it is a continuous process. In some embodiments, after fermentation, the cells are separated from the fermented slurry and re-contacted with a fresh batch of saccharified lignocellulose.

In some embodiments, the fermentation product is separated from the culture using any suitable technique known in the art (e.g., stripping, membrane filtration, and/or distillation), in order to produce purified fermentation product that finds use as a fuel. In some embodiments, the purified fermentation product is present in a concentration in the range of about 5% to about 99.9% (e.g., in the range of about 5% to about 95%, about 10% to about 90%, about 15% to about 85%, about 20% to about 80%, about 25% to about 75%, about 30% to about 70%, about 35% to about 65%, about 40% to about 60%, about 45% to about 55%, or about 50% to 90%). In some embodiments, the purified fermentation product is present in a concentration of about 10 to about 15%. In some embodiments, the fermentation product is ethanol.

### Fermentation Systems

The present invention also provides fermentation systems. In some embodiments, the fermentation system comprising a fermentation tank containing the cell culture described above. In some embodiments, the tank is closed (i.e., a sealed tank), while in other embodiments it is an open tank/system. In some additional embodiments, the system provides anaerobic growth conditions.

In some embodiments, the fermentation system is a batch system, while in other embodiments, it is continuous. A classical batch fermentation is a closed system, wherein the culture is inoculated with the medium at the beginning of the fermentation and no further carbon source is added during fermentation, although factors such as pH and oxygen concentration are typically monitored and modified, as needed. The metabolite and biomass compositions of the batch system change constantly up to the time the fermentation is stopped. Within batch cultures, cells progress through a static lag phase to a high growth log phase and finally to a stationary phase where growth rate is diminished or halted. If untreated, cells in the stationary phase eventually die. In general, cells in log phase are responsible for the bulk of production of end product.

In some embodiments, a “fed-batch fermentation” system in which the carbon source is added in increments as the fermentation progresses, finds use. Fed-batch systems are useful when catabolite repression inhibits the metabolism of the cells and where it is desirable to have limited amounts of substrate in the medium. Measurement of the actual substrate concentration in fed-batch systems is difficult and is therefore estimated on the basis of changes observed in measurable factors such as pH, dissolved oxygen, and the partial pressure of waste gases such as CO₂. Batch and fed-batch fermentations are common and well known to those in the fermentation art.

As indicated above, in some embodiments, continuous fermentation systems find use. In these systems, growth medium is added continuously to a bioreactor and an equal amount of product is simultaneously removed for processing. Continuous fermentation generally maintains the cultures at a constant high density where cells are primarily in log phase.

Continuous fermentation allows for the modulation of one factor or any number of factors that affect cell growth and/or end product concentration. For example, in certain embodiments, a limiting nutrient such as the carbon source or nitrogen source is maintained at a fixed rate and all other parameters are allowed to moderate. In other systems, a number of factors affecting growth can be altered continuously while the cell concentration, measured by media turbidity, is kept constant. Continuous systems strive to maintain steady state growth conditions. Thus, cell loss due to medium being drawn off may be balanced against the cell growth rate in the fermentation. Methods of modulating nutrients and growth factors for continuous fermentation processes as well as techniques for maximizing the rate of product formation are well known to those skilled in the fermentation art.

The foregoing may be better understood in connection with the following non-limiting examples.

### EXPERIMENTAL

The present invention is described in further detail in the following Examples, which are not in any way intended to limit the scope of the invention as claimed.

In the experimental disclosure below, the following abbreviations apply: ppm (parts per million); M (molar); mM (millimolar), uM and μM (micromolar); nM (nanomolar); mol (moles); gm and g (gram); mg (milligrams); ug and μg (micrograms); L and l (liter); ml and mL (milliliter); cm (centimeters); mm (millimeters); um and μm (micrometers); sec. (seconds); min(s) (minute(s)); h(s) (hour(s)); U (units); MW (molecular weight); rpm (rotations per minute); ° C. (degrees Centigrade); DNA (deoxyribonucleic acid); RNA (ribonucleic acid); HPLC (high pressure liquid chromatography); HMF (hydroxymethylfurfural); YPD (Yeast extract 10 g/L; Peptone 20 g/L; Dextrose 20 g/L); propagation medium (160 g/l glucose, 40 g/l xylose, 4.5 g/l arabinose, 20 g/l yeast extract, 6 g/l acetic acid, 0.6 g/l furfural, 0.9 g/l hydroxymethylfurfural with a vitamin solution added to final concentrations of 0.05 mg/l biotin, 1 mg/l calcium pantothenate, 1 mg/l nicotinic acid, 1 mg/l myoinositol, mg/l thiamine chloride hydrochloride, 1 mg/l pyridoxal hydrochloride potassium iodide and a trace element solution added to final concentrations of 0.403 μM EDTA, 15.6 μM ZnSO₄, 5 μM MnCl₂, 1.3 μM CoCl₂, 1.2 μM CuSO₄, 1.6 μM disodium molybdate, 30.6 μM CaCl₂, 10.8 μM FeSO₄, 16.2 μM boric acid, 0.6 μM potassium iodide, 5 g/l NH₄SO₄, 3 g/l K₂PO₄, 0.5 g/l MgSO₄ and pH adjusted to 5.0 with NaOH); ARS (ARS Culture Collection or NRRL Culture Collection, Peoria, Ill.); Lallemand (Lallemand Ethanol Technology, Milwaukee, Wis.); Agilent (Agilent Technologies, Inc., Santa Clara, Calif.); and Bio-Rad (Bio-Rad Laboratories, Hercules, Calif.).

### EXAMPLE 1

### Yeast Fermentation

Frozen glycerol stocks of the yeast strains NRRL YB-1952, NRRL Y7567 (ARS) and the commercial ethanolagens SUPERSTART™ dry yeast, and THERMOSACC® fresh yeast (Lallemand) were inoculated into 50 ml YPD in separate 250 ml glass shake flasks and cultured overnight at 250 rpm and 30° C. Then, 50 mL of each culture was inoculated into 1 L shake flasks containing 200 mL of propagation medium and grown overnight at 250 rpm and 30° C. From these propagation cultures, 400 μL of each strain was dispensed into 24 wells of a deep well microtiter plate and pelleted by centrifugation at 4000 rpm for 10 minutes. Supernatant was decanted and cell pellets resuspended in 400 μL of fermentation medium with the same composition as the propagation medium described above but with acetic acid added to final concentrations of 0, 6, 9 and 12 g/L and pH adjusted to 4, 5 or 6 with NaOH or HCl. The medium was dispensed such that each of the 24 replicate wells of each yeast strain was combined with two replicates of each fermentation medium concentration conditions. Each plate was sealed with non-permeable silicon mats and cell pellets were resuspended by agitation in a microwell plate shaker for 1 minute at room temperature. Fermentation was performed in incubator shakers at 30° C. and 100 rpm for 24 hours. Samples from each well were analyzed with an Agilent 1200 HPLC equipped with a refractive index detector (RID). Glucose, xylose, arabinose, xylitol, xylulose, lactic acid, glycerol, acetic acid, ethanol, HMF and furfural were separated on an ion-exchange column (Aminex HPX-87H; Bio-Rad) at 80° C. Ultrapure water was used as eluent at a flow rate of 0.6 mL/min. The mobile phase and diluent was 0.005M H₂SO₄ at a flow rate of 0.6 mL/min.

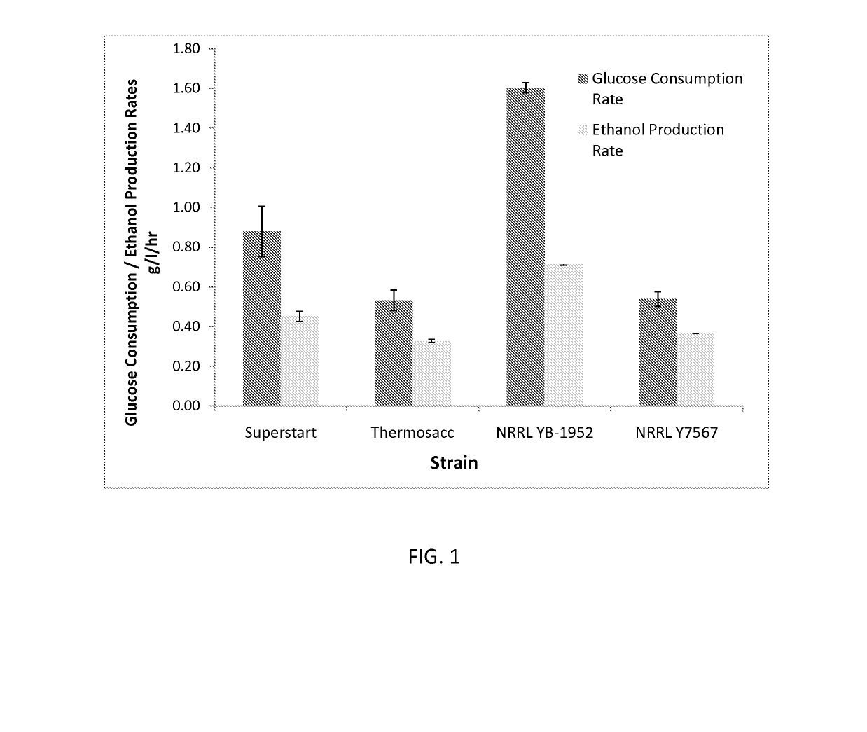

Based on these results, volumetric glucose consumption rates (g/L/hr) were calculated and are presented in FIG. 1. As shown in FIG. 1, NRRL YB-1952 showed significantly higher glucose fermentation rates at low pH, high acetic acid conditions. In addition, this strain produced a higher quantity of ethanol during the fermentations. These results indicate that the fermentation activity of this strain is highly resistant to low pH, high acetic acid conditions, even compared with the commercially available strains SUPERSTART™ dry yeast and THERMOSACC® fresh yeast.

While particular embodiments of the present invention have been illustrated and described, it will be apparent to those skilled in the art that various other changes and modifications can be made without departing from the spirit and scope of the present invention. Therefore, it is intended that the present invention encompass all such changes and modifications with the scope of the present invention.

The present invention has been described broadly and generically herein. Each of the narrower species and subgeneric groupings falling within the generic disclosure also form part(s) of the invention. The invention described herein suitably may be practiced in the absence of any element or elements, limitation or limitations which is/are not specifically disclosed herein. The terms and expressions which have been employed are used as terms of description and not of limitation. There is no intention that in the use of such terms and expressions, of excluding any equivalents of the features described and/or shown or portions thereof, but it is recognized that various modifications are possible within the scope of the claimed invention. Thus, it should be understood that although the present invention has been specifically disclosed by some preferred embodiments and optional features, modification and variation of the concepts herein disclosed may be utilized by those skilled in the art, and that such modifications and variations are considered to be within the scope of this invention.

1. A method for fermentation comprising: providing at least one acetate-resistant Saccharomyces cerevisiae NRRL YB-1952 cell or a genetically modified derivative of said Saccharomyces cerevisiae NRRL YB-1952 cell and a fermentation medium comprising acetate; culturing said at least one acetate-resistant Saccharomyces cerevisiae NRRL YB-1952 cell or a genetically modified derivative of said Saccharomyces cerevisiae NRRL YB-1952 cell in said fermentation medium under conditions such that said at least one acetate-resistant Saccharomyces cerevisiae NRRL YB-1952 cell or a genetically modified derivative of said Saccharomyces cerevisiae NRRL YB-1952 cell produces at least one fermentation product.

2. The method of claim 1, wherein said fermentation medium comprises at least 6 g/L acetate.

3. The method of claim 1, further comprising collecting said fermentation product.

4. The method of claim 3, wherein said collecting comprises distilling said fermentation product from said culture medium.

5. The method of claim 1, wherein said fermentation medium comprises saccharified lignocellulose.

6. The method of claim 1, wherein said fermentation medium comprises lignocellulose feedstock that has been pretreated.

7. The method of claim 6, wherein said saccharified lignocellulose is produced by enzymatic and/or acidic pretreatment of said lignocellulose feedstock.

8. The method of claim 1, wherein said fermentation product is an alcohol.

9. The method of claim 8, wherein said alcohol is ethanol.

10. The method of claim 1, wherein said fermentation medium further comprises at least one organic acid in addition to said acetate.

11. The method of claim 10, wherein said at least one organic acid is present in said fermentation medium at a concentration of at least 6 g/L.

12. The method of claim 1, wherein the fermentation medium has a pH of less than pH 6.0.

13. The method of claim 1, wherein said method is conducted under anaerobic conditions.

14. The method of any claim 1, wherein said method is a batch-fed fermentation method or a continuous fed fermentation method.
